(19) United States
(12) Patent Application Publication
(10) Pub. No.: US 2004/0048779 A1
(43) Pub. Date: Mar. 11, 2004

(54) Use of rotigotine for treating the restless leg syndrome

(76) Inventor: Erwin Schollmayer, Leverkusen (DE)

Correspondence Address: EDWARDS & ANGELL, LLP, P.O. BOX 9169, BOSTON, MA, 02209, US

(21) Appl. No.: 10/429,283

(22) Filed: May 2, 2003

Related U.S. Application Data
(60) Provisional application No. 60/381,509, filed on May 17, 2002.

(30) Foreign Application Priority Data
May 6, 2002 (DE) DE 102 20 230.3

(51) Int. Cl. A61K 38/17

(52) U.S. Cl. 514/2

(57) ABSTRACT

This invention relates to the use of rotigotine for the effective treatment of Restless Leg Syndrome (RLS) as well as to a rotigotine-containing transepicutaneous pharmaceutical composition, provided in particular in the form of an acrylate- or silicone-based Transdermal Therapeutic System (TTS) having a surface area of 2.5 to 20 cm² and containing 0.1 to 3.15 mg/cm² rotigotine as its active component against the Restless Leg Syndrome, said system leading in the human Restless Leg Syndrome condition to an improvement, compared to a placebo treatment, by 2 or more units on the International Restless Leg Syndrome Study Group (IRLSSG) scale after administration over a period of eight days.

This invention relates to the use of rotigotine for the effective treatment of the Restless Leg Syndrome (RLS). The invention also relates to a rotigotine-containing transepicutaneous pharmaceutical composition especially in the form of a Transdermal Therapeutic System (TTS).

### BACKGROUND OF THE INVENTION

The Restless Leg Syndrome (RLS) is a neurological disease that expresses itself as a false sensation in the legs accompanied by a strong kinetic urge. Symptoms of RLS include tingling, pulling, aching, itching, burning, cramps or pain, causing in the person concerned the irresistible urge to move. This disorder occurs most frequently when the person concerned is resting.

It is particularly during the night's sleep that this sensory disorder with its attendant kinetic urge leads to restlessness and sleep interruptions.

RLS can occur at any age but increases in frequency as persons grow older. It afflicts about 10% of the general population. Because of the nature of the symptoms, RLS is one of the most prevalent causes of sleep disturbances. In 20-40 year-olds, RLS accounts for 5%, in 40-60 year-olds for 20% and in those over 60 years of age for 35% of their sleeping-waking problem.

Once the quality of sleep and thus of life of a patient has increasingly deteriorated due to RLS or the patient suffers from daytime somnolence, the need for therapy is indicated. Such need for therapy usually sets in at the age of 40-50.

Therapy studies have revealed a diversity of results obtained in monotherapeutic treatments with dopamine agonists, opiates, benzodiazepines, carbamazepine, clonidine or levodopa (L-DOPA) in combination with a dopa decarboxylase inhibitor. The use of L-DOPA for treating RLS has been the subject of a particularly large number of papers. Long-term L-DOPA therapy leads to a clear mitigation of the disorder with an improved quality of sleep and life. The drawback of L-DOPA therapy, however, lies in the fact that in a great many patients its effectiveness tapers off and/or the RLS problem is shifted toward the morning hours (rebound) or the disorder is aggravated with the problem occurring even during the day (augmentation).

Various dopamine agonists were tested in short-term therapy studies for their therapeutic suitability. The dopamine agonists investigated included bromocriptine, cabergolin, alpha dihydroergocryptine, lisuride, pergolide, pramipexol and ropinirol. All of these dopamine agonists were found to be effective but with a negative aspect in that, usually in the beginning and as a function of the dosage administered, they lead to such side effects as nausea, vomiting, dizziness, hypotension, constipation or insomnia.

Attacks of drowsiness have been described as a serious side effect of pramipexol.

Benzodiazepines and opiates as well are effective against RLS. However, because of the risk of an addiction and progressive tolerance these substances are suitable for therapeutic application to a limited extent at best.

Also examined for its effectiveness in the treatment of RLS was clonidine, 2-(2,6-dichloroanilino)-4,5-dihydroimidazol, originally developed as an antihypertensive agent and miotic. While it was found that soporiferous latency was reduced, it had no effect on the quality of sleep, the frequency of waking up or periodic leg movement during sleep (“PLMS”). Given that more efficacious substances are available for monotherapy, clonidine is not currently recommended as an alternative form of therapy except in limited situations.

The drawback of most conventional monotherapies is that, depending on the duration of the therapy, the amount of the active ingredient must be progressively increased in order to ensure the success of the treatment.

A surprising discovery has shown that the monotherapeutic administration of a rotigotine-containing transepicutaneous composition especially when in the form of a pharmaceutical TTS composition leads to the suppression and reduction of the RLS symptoms, with rotigotine as the active substance requiring only very small doses compared to conventional monotherapies.

### DESCRIPTION OF THE INVENTION

This invention relates to the use of rotigotine in producing medications for the effective treatment of the Restless Leg Syndrome while avoiding the known drawbacks of traditional current-art monotherapies.

Rotigotine is the INN (International Nonproprietary Name) of the chemical substance (−)-5, 6, 7, 8-tetrahydro-6-[propyl [2-(2-thienyl)ethyl]amino]-1-naphtol.

The invention further relates to a rotigotine-containing pharmaceutical composition in the form of a transepicutaneous pharmaceutical preparation and especially in the form of a Transdermal Therapeutic System (TTS) that avoids the current-art drawbacks traditionally encountered in monotherapy with orally administered drugs.

It is a known fact that (−)-5, 6, 7, 8-tetrahydro-6-[propyl [2-(2-thienyl)ethyl]amino]-1-naphtol can be used in the treatment of Parkinson's disease.

It has now been found that administering (−)-5, 6, 7, 8-tetrahydro-6-[propyl [2-(2-thienyl)ethyl]amino]-1-naphtol has a very beneficial effect on the disease known as Restless Leg Syndrome.

Surprisingly, the compound to be used according to this invention already shows to be clearly effective even in very low dosages, making it suitable for use in the treatment of Restless Leg Syndrome. Both in terms of its effective strength and its specificity this compound is superior to the substances that have so far been known to be effective when the treatment of Restless Leg Syndrome was indicated.

It lends itself particularly well to the treatment of moderate to severe cases of the RLS disease.

The prior-art lineup of monotherapy agents for treating RLS has not so far included anything with that strong and specific an effect, making the use of this inventive compound for the treatment of Restless Leg Syndrome an enriching addition to the fields of pharmaceutics and medicine.

The (−)-5, 6, 7, 8-tetrahydro-6-[propyl [2-(2-thienyl)ethyl]amino]-1-naphtol compound is prepared in essentially conventional fashion. This compound is prepared employing the method described in EP 0 168 505 B1. For the purpose of this invention, reference is made to that document in its entirety.

The advantage offered by the invention lies in the fact that the use of rotigotine as the active ingredient for treating Restless Leg Syndrome allows even low dosages to improve the patient's condition without causing intolerable, undesirable effects (side effects). As an important feature, any augmentation is suppressed. It also improves the responsiveness and response rate of the RLS patients. Within the scope of this invention, the active substance is advantageously applied in quantities of 1.0 to 10 mg and preferably 0.5 to 5 mg per day.

The active substance is applied on the patient's skin in transepicutaneous form as an ointment, a gel or a cream, but preferably as a TTS in the form of a patch.

According to the invention, the Transdermal Therapeutic System contains a backing that is inert relative to the matrix content, a self-adhesive matrix layer containing (−)-5, 6, 7, 8-tetrahydro-6-[propyl [2-(2-thienyl)ethyl]amino]-1-naphtol, and a protective film that must be peeled off before use, the system being characterized in that the matrix layer

a) includes a substrate layer of a non-aqueous acrylate- or silicone-based polymer adhesive,

b) features a solubility of ≧5% (g/g) for the free base (−)-5, 6, 7, 8-tetrahydro-6-[propyl [2-(2-thienyl)ethyl]amino]-1-naphtol, and

c) contains said free base (−)-5, 6, 7, 8-tetrahydro-6-[propyl [2-(2-thienyl)ethyl]amino]-1-naphtol in an effective amount.

In an enhanced variation, the TTS matrix contains <0.5% (g/g) of inorganic silicate particles.

In a particularly desirable version the matrix of the Transdermal Therapeutic System contains <0.05% (g/g) of inorganic silicate particles.

In one embodiment of the invention, the Transdermal System includes an acrylate-based polymer adhesive that contains at least two of the following monomers:

Acrylic acid, acrylamide, hexyl acrylate, 2-ethylhexyl acrylate, hydroxyethyl acrylate, octyl acrylate, butyl acrylate, methyl acrylate, glycidyl acrylate, methacrylic acid, methacrylamide, hexylmethyl acrylate, 2-ethylhexyl methacrylate, octyl methacrylate, methylmethacrylate, glycidyl methacrylate, vinyl acetate or vinyl pyrrolidone.

In an advantageously enhanced version of the invention the Transdermal System includes a silicone-based polymer adhesive with additives that improve the solubility of (−)-5, 6, 7, 8-tetrahydro-6-[propyl [2-(2-thienyl)ethyl]amino]-1-naphtol, in the form of hydrophilic polymers or glycerin or glycerin derivatives.

In another implementation of the invention, the Transdermal System (−)-5, 6, 7, 8-tetrahydro-6-[propyl [2-(2-thienyl)ethyl]amino]-1-naphtol includes a polymer adhesive at a concentration of 10 to 35% [g/g] if acrylate-based or of 5 to 40% [g/g] if silicone-based.

In another enhanced version of the invention, the Transdermal System contains substances that improve the permeation of (−)-5, 6, 7, 8-tetrahydro-6-[propyl [2-(2-thienyl)ethyl]amino]-1-naphtol into the human skin.

According to the invention, the permeation-promoting substance added to the Transdermal System is selected from among the lipidols, fatty acids, fatty acid esters, fatty acid amides, glycerin or its derivatives, N-methylpyrrolidone, terpenes or terpene derivatives.

In one implementation of the invention, the permeation-promoting substance in the Transdermal Therapeutic System id oleic acid or oleyl alcohol.

According to the invention, a desirable hydrophilic polymer in the Transdermal Therapeutic System is polyvinyl pyrrolidone, a copolymer of vinyl pyrrolidone and vinyl acetate, polyethylene glycol, polypropylene glycol or a copolymer of ethylene and vinyl acetate.

In another implementation, the hydrophilic polymer contained in the Transdermal Therapeutic System in the form of soluble polyvinyl pyrrolidone at a concentration of 1.5 to 5% (g/g) within the drug-containing matrix layer.

According to the invention, the matrix of the Transdermal System can additionally contain inert, cohesion-improving fillers.

The method for producing a Transdermal Therapeutic System according to the invention includes the following procedural steps:

i) Mixing of a suspension of (−)-5, 6, 7, 8-tetrahydro-6-[propyl [2-(2-thienyl)ethyl]amino]-1-naphtol hydrochloride in ethanol with an alkaline compound in ethanol for converting the hydrochloride into its free base;

ii) Filtering of the resulting suspension if and as necessary;

iii) Addition of polyvinyl pyrrolidone and an adhesive solution; and

iv) Drying of the product.

According to one implementation of the invention, the alkaline compound used in this process is sodium hydroxide or potassium hydrate.

In another implementation of the invention, the alkaline compound employed is sodium- or potassium metasilicate or trisilicate.

Finally, as part of the process prior to the drying of the product, the mixture can be coated onto an inert backing or protective film in such fashion as to produce a uniform layer.

As a result, the product obtained by the method described offers any one or a combination of the aforementioned features.

The Transdermal Therapeutic System can be produced as described in detail in the implementation examples of EP 1 033 978 B1.

A pharmaceutical product per this invention includes a reinforcing layer that is inert relative to the constituent components of the matrix, a self-adhesive matrix layer that contains an effective amount of rotigotine or rotigotine hydrochloride, and a protective film that must be removed before the product is used.

### IMPLEMENTATION EXAMPLE #1

A Polyacrylate System with (−)-5, 6, 7, 8-tetrahydro-6-[propyl [2-(2-thienyl)ethyl]amino]-1-naphtol

Added to a 264 g solution of a polyacrylate adhesive with a 50% solids content is a 66 g amount of a 50% Eudragit E100 solution in ethyl acetate, followed by the addition of 36 g oleyl alcohol and homogenization of the compound by agitation.

Next, 89.65 g of (−)-5, 6, 7, 8-tetrahydro-6-[propyl [2-(2-thienyl)ethyl]amino]-1-naphtol is dissolved in 200 ml methylethyl ketone and stirred into the above compound. Once the compound has been homogenized, it is coated by means of a suitable doctor blade onto a siliconized polyester film. The thickness of the moist layer is so gauged that after removal of the solvent and 30 minutes of drying at 50° C. the resulting weight of the layer is 60 g/m².

Next, the dried matrix layer is backed with a polyester film 13 μm thick. From the laminated patch material thus obtained, finished patches are punched out in the desired sizes and packed in packaging bags.

The (−)-5, 6, 7, 8-tetrahydro-6-[propyl [2-(2-thienyl)ethyl]amino]-1-naphtol is present in the patch matrix at a concentration of 30.8%. Examples of suitable polyacrylate adhesives include Durotak 387-2051, Durotak 387-2287, Durotak 387-2353, and Durotak 387-2516, all from National Starch & Chemical.

### IMPLEMENTATION EXAMPLE #2

Silicone System with (−)-5, 6, 7, 8-tetrahydro-6-[propyl [2-(2-thienyl)ethyl]amino]-1-naphtol

Added to 24 g of a 25% solution of Kollidon 90F is an 18 g amount of (−)-5, 6, 7, 8-tetrahydro-6-[propyl [2-(2-thienyl)ethyl]amino]-1-naphtol dissolved in 40 g ethanol, followed by homogenization of the compound. Next, 251 g of a solution of an amine-resistant silicone adhesive with a 70% solids content is added to the compound which is then homogenized by continued agitation.

Next, the compound is coated with a suitable doctor blade onto an adhesive polyester film (Scotchpak 1022) with a layer thickness so gauged that after removal of the solvents by 30 minutes of drying at 50° C. the resulting weight of the layer is 50 g/m².

Next, the dried matrix layer is backed with a polyester film 13 μm thick. From the laminated patch material thus obtained, finished patches are punched out in the desired sizes and packed in packaging bags.

The (−)-5, 6, 7, 8-tetrahydro-6-[propyl [2-(2-thienyl)ethyl]amino]-1-naphtol base is present in the patch matrix at a concentration of 9%.

Examples of suitable amine-resistant silicone adhesives include BIO-PSA Q7-4301 and BIO-PSA Q7-4201, both from Dow Corning.

IMPLEMENTATION EXAMPLE #3

A 25 g amount of (−)-5, 6, 7, 8-tetrahydro-6-[propyl [2-(2-thienyl)ethyl]amino]-1-naphtol hydrochloride, together with 14.7 g sodium metasilicate or 16.8 g sodium trisilicate, is agitated in 40 ml ethanol for 48 hours at room temperature. Optionally, the active solution is then filtered followed by the addition of 9.2 g oleyl alcohol, 63.2 g of a 52% polyacrylate adhesive solution (Durotak 387-2287 by National Starch & Chemical) and 22.8 g of a 40% (g/g) Eudragit E100 solution (by Röhm Pharma), whereupon the compound is homogenized by mechanical agitation.

Next, the patch matrix is produced by coating the compound onto a suitably adhesive film and the solvents are removed by 20 minutes of drying at 50° C. The weight of the applied dried matrix layer is around 80 g/m².

The dried matrix layer is backed with a polyester film 23 μm thick. The individual patches are then punched out of the laminated sheet.

Finally, according to the invention, it is particularly desirable to use a silicone-based Transdermal Therapeutic System whose main component consists of at least one imperatively amine-resistant silicone compound.

The silicone compound is usually a pressure-sensitive adhesive or a corresponding mixture, forming a matrix in which other TTS components are embedded. The adhesive(s) should preferably also be pharmaceutically acceptable, meaning biocompatible, nonsensitizing and nonirritant on the skin. Particularly desirable silicone-based adhesives for use according to the invention should also meet the following requirements:

Adhesive and cohesive properties unaffected by moisture or perspiration within normal temperature fluctuations;

Good compatibility with rotigotine and with other carrier substances used in the formulation; in particular, the adhesive should not react with the amine group of rotigotine.

According to the invention it has been found that pressure-sensitive adhesives of the type constituting a soluble, polycondensed polydimethyl siloxane (PDMS)/resin network, with the hydroxy end groups covered for instance by trimethylsilyl (TMS) groups, are particularly useful. Preferred adhesives of this type include the pressure-sensitive BIO-PSA silicone adhesives produced by Dow Corning, especially the Q7-4201 and Q7-4301 grades. However, other silicone-based adhesives can also be used.

For that same purpose, the invention further and preferably provides for a silicone-based Transdermal Therapeutic System whose primary adhesive components comprise two or more silicone resins. It may be desirable for such a silicone adhesive mixture to be composed of at least one strongly adhesive substance and at least one moderately adhesive substance so as to ensure an optimal balance between strong adhesion and a low level of cold flux. Excessive cold flux can cause a patch to be too soft, easily sticking to the packing material or to the patient's clothing. Moreover, this mixture of adhesives would appear to be particularly useful in producing an effective Transdermal Therapeutic System. According to the invention, a mixture found to be especially well suited consists of roughly equal amounts of the aforementioned amine-resistant, pressure-sensitive silicone-based adhesives Q7-4201 (moderate adhesion) and Q7-4301 (strong adhesion).

In another preferred form of implementation the silicone-based Transdermal Therapeutic System includes a solubilizer. A variety of tenside-like or amphiphilic substances can serve as solubilizers. They should be pharmaceutically acceptable and approved for use in medicines. Advantageously, the solubilizer would also have the ability to enhance the cohesion of the Transdermal Therapeutic System. One particularly preferred example of such a solubilizer is soluble polyvinyl pyrrolidone. Polyvinyl pyrrolidone is commercially available, for instance under the tradename Kollidon® (Bayer AG). Other examples include copolymers of polyvinyl pyrrolidone and vinyl acetate, polyethylene glycol, polypropylene glycol, glycerin and fatty acid esters from glycerin or copolymers of ethylene and vinyl acetate.

The silicone-based Transdermal Therapeutic System for use according to the invention contains less than 1% by weight of inorganic silicates and is most preferably completely free of any inorganic silicates.

The water content of the Transdermal Therapeutic System for use according to the invention is preferably low enough to obviate the need for evaporation during the TTS production process. Typically, the water content of a newly produced patch is less than 2% by weight and preferably 1% by weight or below that.

In a particularly preferred form of implementation of the invention the Transdermal Therapeutic System extends over 10 to 30 cm² and preferably 5 to 20 cm². Needless to say, a TTS with an area for instance of 20 cm² is pharmacologically equivalent to two 10 cm² patches or four 5 cm² patches that contain the same drug amount per cm², and is correspondingly interchangeable with these. Accordingly, the patch sizes mentioned in this patent application are to be understood to refer to the entire surface of all TTS's that are simultaneously applied on a patient at any one time.

The provision and application of one or several Transdermal Therapeutic Systems according to the invention offer a pharmacological advantage over an oral therapy in that the treating physician can determine and apply the optimal dose for each patient relatively quickly and with specificity and precision simply by selecting or increasing the number or size of the patches given the patient.

If a seven-day patch is desired, it is generally necessary to select a higher drug content. It has been found that a rotigotine content in the range approximately from 0.4 to 0.5% by weight is particularly advantageous since it provides for optimal utilization of the medication contained in the TTS, i.e. after the administration the residual drug amount in the TTS is quite small. The dose administered via such a TTS is usually 50% or more of the amount of medication originally contained in the TTS and may even be as high as 80 to 90%.

The fact that the silicone-based Transdermal Therapeutic System described in this invention has a significant therapeutic effect on the symptoms of Restless Leg Syndrome even with area sizes of 10 to 20 cm² or especially even with those less than 10 cm² and a low drug content of about 0.4 to 0.5 mg/cm², especially 0.45 g/cm², must be viewed as another useful feature of the invention.

The Transdermal Therapeutic System employed in accordance with the invention is preferably a patch with a continuously adhesive matrix containing the medication at least in its central area. However, according to the invention the concept also includes transdermal equivalents of such patches, meaning a form of implementation in which the medication is located in an inert, nonadhesive central region of the TTS while an adhesive section extends along the perimeter of the patch.

This invention further relates to a method for treating the RLS disease in that a Transdermal Therapeutic System with a surface size of 5 to 20 cm² is applied on a patient.

The following is a detailed description of the invention and of the optimal form of its implementation.

### IMPLEMENTATION EXAMPLE #4

A Transdermal Therapeutic System employing a combination of pressure-sensitive silicone-based adhesives was produced as follows:

(−)-5, 6, 7, 8-tetrahydro-6-[propyl [2-(2-thienyl)ethyl]amino]-1-naphthalenol hydrochloride (rotigotine hydrochloride at 150 g) was added to a solution of 17.05 g NaOH in 218 g ethanol (96%). The resulting mixture was agitated for about 10 minutes. This was followed by the addition of 23.7 g sodium phosphate buffer solution (8.35 g Na₂HPO₄×2H₂O and 16.07 g Na₂PO₄×2H₂O in 90.3 g water). Insoluble or precipitated substances were removed from the mixture by filtration. The filtrate was washed with 60.4 g ethanol (96%) in order to obtain a particle-free ethanol solution of rotigotine in its free-base form.

The solution of rotigotine in its free-base form (346.4 g) in ethanol (35% g/g) was mixed with 36.2 g ethanol (96%). The resulting solution was mixed with 109 g of an ethanol solution containing 25% by weight of polyvinyl pyrrolidone (KOLLIDON® 90F), 0.077% by weight of a liquid sodium bisulfide solution (10% by weight), 0.25% by weight of ascorbyl palmitate and 0.63% by weight of DL-alpha-tocopherol until homogeneity was attained. This was followed by the addition, to the mixture thus obtained, of 817.2 g of an amine-resistant, strongly adhesive silicone substance (BIO-PSA® Q7-4301 manufactured by Dow Corning (74% by weight solution in heptane), 851.8 g of an amineresistant moderate-adhesion silicone adhesive (BIO-PSA® Q7-4201 manufactured by Dow Corning) (71% by weight solution in heptane), and 205.8 g ligroin (heptane), whereupon all components were agitated until a homogeneous dispersion was obtained.

The dispersion was applied, with an appropriate physician's knife, on a suitable release film of polyester (SCOTCHPAK® 1022), and the solvents were continuously removed in a drying oven at temperatures of up to 80° C. for about 30 minutes, thus yielding a medication-containing adhesive matrix with a layer weight of 50 g/m². The dried matrix layer was laminated with a polyester-type reinforcing film (SCOTCHPAK® 1109). The individual patches were punched out of the completed laminates in the desired sizes (e.g. 5 cm², 10 cm², 20 cm², 30 cm²) and sealed in bags under a nitrogen-flux atmosphere.

The following table shows the composition, in mg/20 cm², of an inventive Transdermal Therapeutic System containing a combination of silicone-type PSA.

| Constituents of the Composition | Quantity (mg) |
|---|---|
| Rotigotine base | 9.00 |
| Polyvinyl pyrrolidone | 2.00 |
| Silicone BIO-PSA ® Q7-4301 | 44.47 |
| Silicone BIO-PSA ® Q7-4201 | 44.46 |
| Ascorbyl palmitate | 0.02 |
| DL-alpha tocopherol | 0.05 |
| Sodium metabisulfite | 0.0006 |
| Weight of matrix layer | 50 g/m² |

Clinical Tests

The rotigotine TTS produced per one of the above implementation examples was investigated in placebo-controlled, double-blind, randomized clinical tests at several centers, covering in a study with triple-branched parallel groups 63 patients who were suffering from moderate to very severe idiopathic Restless Leg Syndrome conditions.

The average age of the patients was 58.3 years. Randomization in the three treatment groups in terms of gender, age and severity of prior illnesses was reasonably balanced.

After gradual and complete termination of the preceding L-dopa treatment and a 7±4-day break in the therapy (washout) the patients were treated with the rotigotine TTS.

Over a treatment period of eight (8) days the patients of one group were treated with 5 cm² TTS and the patients of another group were treated with 10 cm² TTS. For a comparison the patients in the placebo group were treated with a placebo TTS. Within 24 hours, each of the above-mentioned drug-containing TTS's released 2.25 mg and, respectively, 4.5 mg rotigotine to the patient concerned.

As it turned out, a significant alleviation of the symptoms of Restless Leg Syndrome on patients that had been suffering from that disorder already manifested itself after only one (1) week. At that point in time the patients were not taking any other medicine against RLS.

As a result of the prespecified primary effectiveness the activities of daily life and the motoricity had changed, based on the generally accepted rating scale of the International Restless Leg Syndrome Study Group (IRLSSG), from the initial value to that in the concluding treatment evaluation (8^{th} day).

On the basis of 10 questions, the IRLSSG measures and rates the following clinical parameters in Restless Leg patients:

1. On the following scale, what is your assessment of the overall level of severity of the RLS condition in your legs and arms?

0=none—no symptoms

1=minor

2=moderate

3=elevated

4=very severe symptoms

2. In general, how strong in your estimation is your RLS-related urge to move?

0=none felt

1=minor

2=moderate

3=elevated

4=very strong

3. Overall, to what degree has movement helped to alleviate the RLS problem in your arms and legs?

0=there were no RLS-related problems to be alleviated

1=completely or almost completely

2=moderately

3=a little

4=not at all

4. To what extent has your sleep been interrupted by RLS problems?

0=not at all

1=minor

2=moderate

3=to quite an extent

4=very much

5. How tired or sleepy were you during the day due to your RLS problem?

0=not at all

1=a little

2=moderately

3=to a fair degree

4=very much so

6. Overall, what was the severity of your RLS problems?

0=zero

1=minor

2=moderate

3=elevated

4=very high

7. How often did your RLS problems occur?

0=never

1=from time to time (i.e. on 1 day during the last 7 days)

2=occasionally (i.e. on 2 to 3 out of the last 7 days)

3=frequently (i.e. on 4 to 5 out of the last 7 days)

4=very often (i.e. on 6 to 7 out of the last 7 days)

8. When you had RLS problems, what was their average severity?

0=none

1=minor (i.e. for less than 1 hour during a 24-hour day)

2=moderate (i.e. for 1 to 3 hours during a 24-hour day)

3=elevated (i.e. for 3 to 8 hours during a 24-hour day)

4=very high (i.e. for 8 or more hours during a 24-hour day)

9. To what degree, in general, have your RLS-related problems affected your ability to go about your daily activities such as a satisfactory family, private, scholastic or working life?

0=not at all

1=slightly

2=moderately

3=to a fair degree

4=very much

10. How much did your RLS-related problems affect your mood, for example, were you angry, depressed, sad, anxious or irritable?

0=not at all

1=slightly

2=moderately

3=to a fair extent

4=very much

The overall IRLSSG rating is derived from the individual values as follows:

First, an initial starting value is determined for each patient participating in the study. This is done by adding up the individual IRLSSG parameter values as of Day 0, i.e. before the treatment. Over the course of the treatment the IRLSSG values are compared with the starting value and any changes from that starting value are recorded. Finally, the average improvement of the IRLSSG value as of Day 8 over the starting value is determined by calculating the average of all test subjects. The resulting value is called CAS—(complete analysis set) randomized average change from the starting value of the overall IRLSSG rating. The term “randomized” indicates that in terms of their different prespecified dosages the patients had been subjected to prior double-blind randomization.

Patients suffering from the Restless Leg Syndrome have been known to display a relatively strong response to placebos, meaning that even when treated with placebos these Restless Leg Syndrome patients show a certain improvement of their IRLSSG values. It is therefore important to compare any effect of the drug treatment with the degree of the IRLSSG improvement obtained through placebo treatment over the same time period. Thus, the final assessment of the improvement takes into account the effect of a placebo treatment during the same time period.

Results

Between the starting value and that established 8 days after the application of the TTS per this invention, a significant dosage-related improvement of the IRLSSG values was recorded in comparison with the placebo group, especially in the 4.5 g group. That result is reflected in the following table.

| | | Reduction of the average | |
|---|---|---|---|
| | | IRLSSG Value as compared | |
| Patch Size | Amount of | to Placebo Treatment by | |
| (TTS) | Rotigotine | Day 8 | p (one side) |
| 5 cm² | 2.25 mg | 3.6 | 0.09 |
| 10 cm² | 4.5 mg | 6.3 | 0.04 |

The value referred to as “p” in the above table represents the one-sided p-value arrived at by a statistical evaluation of the test data.

At the end of the 8-day treatment both patient groups reported that nearly all subjective symptoms such as tingling, cramps and pain in the legs, restlessness in the legs during the night, and difficulties falling or remaining asleep had either disappeared or diminished to a tolerable minimum and no longer had a negative effect on their daily quality of life.

The patients also reported that, as a function of the active-substance dose administered to them, the treatment did not or only to a very minimal extent cause any daytime weariness, nausea, dizziness, vomiting of insomnia etc.

Rotigotine, when administered via the TTS according to the invention, was generally well tolerated.

Skin reactions at the point of application were generally very minor.

Conclusions

The above results are the first of their kind to show in a double-blind placebo-controlled study that a dopamine agonist (rotigotine), when transdermally administered once daily, is well tolerated and safe and leads to a distinct clinical improvement in patients in a moderate to severe stage of Restless Leg Syndrome.

No such result has so far been attainable with medications orally administered in monotherapy. Even an improvement of the IRLSSG value of 2 points compared to a placebo can be considered a success. An improvement by over 3 or 6 and more rating units constitutes an all the more significant therapeutic advance and is therefore the preferred objective of this invention.

1. Use of (−)-5, 6, 7, 8-tetrahydro-6-[propyl [2-(2-thienyl)ethyl]amino]-1-naphtol for the treatment of Restless Leg Syndrome.

2. Use of (−)-5, 6, 7, 8-tetrahydro-6-[propyl [2-(2-thienyl)ethyl]amino]-1-naphtol in producing a medication for the transepicutaneous treatment of the Restless Leg Syndrome.

3. Use of (−)-5, 6, 7, 8-tetrahydro-6-[propyl [2-(2-thienyl)ethyl]amino]-1-naphtol in producing a transdermal therapeutic system (TTS).

4. Use as in claims 1 to 3, characterized in that said medication is suitable for the application of 0.5 to 10 mg of the agent per day.

5. Transdermal therapeutic system comprising a backing that is inert relative to the components of the matrix, a self-adhesive matrix layer containing (−)-5, 6, 7, 8-tetrahydro-6-[propyl [2-(2-thienyl)ethyl]amino]-1-naphtol, and a protective film that is to be removed prior to use, characterized in that the matrix
a) includes as a substrate a non-aqueous, acrylate- or silicon-based polymer adhesive,
b) has a solubility of ≧5% (g/g) for its free base (−)-5, 6, 7, 8-tetrahydro-6-[propyl [2-(2-thienyl)ethyl]amino]-1-naphtol, and
c) contains the free base (−)-5, 6, 7, 8-tetrahydro-6-[propyl [2-(2-thienyl)ethyl]amino]-1-naphtol in an effective quantity.

6. Transdermal therapeutic system as in claim 5, characterized in that the matrix contains <0.5% (g/g) inorganic silicate particles.

7. Transdermal therapeutic system as in claim 5, characterized in that the matrix contains <0.05% (g/g) inorganic silicate particles.

8. Transdermal system as in claim 5, in which the acrylate-based polymer adhesive contains at least two of the following monomers:
Acrylic acid, acrylamide, hexyl acrylate, 2-ethylhexyl acrylate, hydroxyethyl acrylate, octyl acrylate, butyl acrylate, methyl acrylate, glycidyl acrylate, methacrylic acid, methacrylamide, hexylmethyl acrylate, 2-ethylhexyl methacrylate, octyl methacrylate, methylmethacrylate, glycidyl methacrylate, vinyl acetate or vinyl pyrrolidone.

9. Transdermal system as in claim 5, in which the silicone-based polymer adhesive contains additives in the form of hydrophilic polymers or glycerin or glycerin derivatives serving to improve the solubility of (−)-5, 6, 7, 8-tetrahydro-6-[propyl [2-(2-thienyl)ethyl]amino]-1-naphtol.

10. Transdermal system as in claim 8 or 9, in which (−)-5, 6, 7, 8-tetrahydro-6-[propyl [2-(2-thienyl)ethyl]amino]-1-naphtol is contained in the acrylate-based polymer adhesive at a concentration of 10 to 35% [g/g] or in the silicone-based polymer adhesive at a concentration of 5 to 40% [g/g].

11. Transdermal system as in claim 10, containing substances that serve to enhance the permeation of (−)-5, 6, 7, 8-tetrahydro-6-[propyl [2-(2-thienyl)ethyl]amino]-1-naphtol into the human skin.

12. Transdermal system as in claim 11, in which the permeation-promoting substance is selected from the group comprising lipidols, fatty acids, fatty acid esters, fatty acid amides, glycerin or its derivatives, N-methylpyrrolidone, terpenes or terpene derivatives.

13. Transdermal system as in claim 12, in which the permeation-promoting substance is oleic acid or oleyl alcohol.

14. Transdermal system as in claim 9, in which the hydrophilic polymer is polyvinyl pyrrolidone, a copolymer of vinyl pyrrolidone and vinyl acetate, polyethylene glycol, polypropylene glycol or a copolymer of ethylene and vinyl acetate.

15. Transdermal system as in claim 14, in which the hydrophilic polymer is soluble polyvinyl pyrrolidone present in the drug-containing matrix layer at a concentration of 1.5-5% (g/g).

16. Transdermal system as in claim 5, in which the matrix contains inert fillers serving to enhance cohesion.

17. Method for producing a transdermal therapeutic system, encompassing the following procedural steps:
i) Mixing of a suspension of (−)-5, 6, 7, 8-tetrahydro-6-[propyl [2-(2-thienyl)ethyl]amino]-1-naphtol hydrochloride in ethanol with an alkaline compound in ethanol for converting the hydrochloride into its free base;
ii) Filtering of the resulting suspension if and as necessary;
iii) Addition of polyvinyl pyrrolidone and an adhesive solution; and
iv) Drying of the product.

18. Method as in claim 17 whereby the alkaline compound employed is sodium hydroxide or potassium hydrate.

19. Method as in claim 17, whereby the alkaline compound employed is sodium- or potassium silicate or trisilicate.

20. Method as in claim 17, whereby prior to the drying of the product the mixture is coated onto an inert backing or protective film in such fashion as to produce a uniform layer.

21. Product obtained by a method per one of the claims 17 to 20.

22. Use of a silicone-based transdermal therapeutic system with a surface area of 5 to 20 cm² and containing 0.1 to 3.15 mg/cm² rotigotine as its active component, for producing an anti-Restless Leg Syndrome medication which in a human patient afflicted with Restless Leg Syndrome brings about an improvement, compared to a placebo treatment, by 2 or more units on the International Restless Leg Syndrome Study Group (IRLSSG) scale after at least eight days of application.

23. Method for treating the Restless Leg Syndrome by applying a transdermal therapeutic system having a surface area of 5 to 20 cm² and containing 0.1 to 3.15 mg/cm² rotigotine as its active component, on a patient afflicted with that disease, leading to an amelioration of the patient's condition by about 2 or more units on the Restless Leg Syndrome Study Group scale, as compared to a placebo treatment, after administration over a time period of 8 days.

24. Transdermal therapeutic system for treating the Restless Leg Syndrome, having a size of 5 to 20 cm² and containing in its matrix 0.4 to 0.5 mg/cm² rotigotine as its active component, said matrix comprising primarily a mixture of at least two amine-resistant silicone-based adhesives.
